(19) United States
(12) Patent Application Publication
(10) Pub. No.: US 2005/0112194 A1
(43) Pub. Date: May 26, 2005

(54) Micronutrient supplement dispensing package

(75) Inventors: Eric Gervais, Laval (CA); Gordana Atanackovic, Dollard-des-Ormeaux (CA)

Correspondence Address: FULBRIGHT & JAWORSKI L.L.P., 600 CONGRESS AVE., SUITE 2400, AUSTIN, TX, 78701, US

(73) Assignee: Duchesnay Inc., Laval, QC (CA)

(21) Appl. No.: 10/922,800

(22) Filed: Aug. 20, 2004

(30) Foreign Application Priority Data
Aug. 21, 2003 (CA) 2,438,160

(51) Int. Cl. A61K 9/48; A61K 9/20; A61K 33/26

(52) U.S. Cl. 424/464; 424/646; 206/531

(57) ABSTRACT

Provided herein is a prenatal and postpartum multivitamin and mineral supplement package provided in the form of a plurality of solid oral dosage units individually contained in blister packs wherein a portion of the dosage forms are iron-containing while at least half of the dosage units are essentially iron-free and wherein the total amount of iron in the package as sold to purchasers is less than about 1300 mg of elemental iron.

This application claims priority to co-pending Canadian Application No. 2,438,160 filed Aug. 21, 2003. The entire text of the above application is incorporated by reference.

### FIELD OF THE INVENTION

The present invention relates to a micronutrient supplement dispensing package. More specifically, the present invention is concerned with a micronutrient supplement dispensing package provided with safety features so as to avoid lethal or deleterious toxicity to a child should a child accidentally ingest the contents of the package as sold.

### BACKGROUND OF THE INVENTION

Micronutrient compositions are commonly taken as dietary aids; either as therapeutic preparations directed to a specific medical problem or as general nutritional supplements. Micronutrients may be broadly defined as substances that are essential or helpful for the maintenance of normal or enhanced metabolic function, but are not normally or sufficiently synthesized in the body and must thus be supplied from an exogenous source.

Given poor dietary habits of individuals and other factors, it has become clear that the role of micronutrient compositions is substantial when it comes to preventing fatigue, disease and optimizing cell maintenance and development. This is particularly the case for individuals who lead a stressful lifestyle, for pregnant women or those who engage in a large amount of physical exercise. Additionally, many drugs, some chronic diseases (e.g. rheumatoid arthritis), certain cancer treatments, and alcoholism can all lead to a deficiency in one or more micronutrients.

It is has also been suggested that a significant portion of preventable illnesses (which it is estimated absorbs as much as 70 percent of total health care costs in the United States) could be readily prevented through supplementing the diet with micronutrients. In addition to major health care cost savings other benefits of supplementation include better quality of life, longer life, and increased productivity. The level of supplements required for effective disease protection cannot be obtained through even the most healthful diet (Bendich, Adrianne, et al. Potential health economic benefits of vitamin supplementation. Western Journal of Medicine, Vol. 166, May 1997, pp. 306-12).

Micronutrients, including multivitamins and mineral supplements are especially important to pregnant or lactating women, ensuring an adequate provision of nutrients for the developing fetus and for the mother. It has become clear that the role of micronutrients is substantial when it comes to preventing fatigue, disease and optimizing cell maintenance and development.

However, one of the leading causes of preventable deaths among toddlers is the accidental ingestion of iron-containing micronutrient supplements such as vitamins and mineral supplements.
Source: The Merck Manual of Diagnosis and Therapy, 16^{th} edition, 1992, page 2128 Pediatrics and Genetics, Injuries, Poisonings and Resuscitation: under the heading “Iron Poisoning”,
“The oral lethal dose of elemental Iron (Fe) is from 200 to 250 mg/kg, but as little as 130 mg of elemental Fe has been fatal.”
Also see the Juurlink et al. “Iron poisoning in young children: association with the birth of a sibling”, Canadian Medical Association Journal, Jun. 10, 2003, 168(12), in the Abstract:
“Iron is a leading cause of death due to poisoning in young children. Because perinatal iron therapy is common, the presence of these tablets, which have a candylike appearance, in the home may pose a hazard to a mother's other young children.”

Pregnancy multivitamins and mineral supplements are particularly dangerous as they contain large amounts of iron. Typical prenatal products contain 60 mg of elemental iron per tablet. Juurlink et al., precited.

However, iron is an important ingredient of pregnancy supplements so as to prevent iron sufficiency and anemia during pregnancy. Iron insufficiency and anemia are characterized by poor transport of oxygen to tissues throughout the body via hemoglobin and myoglobin.

Toddlers are particularly at risk since they are by nature inquisitive, resourceful and capable of opening multivitamin containers. They tend to imitate gestures such as taking vitamins. Toddlers are also particularly at risk because of their sensitivity to iron poisoning. This sensitivity decreases with age.

Strikingly, a single bottle of the leading pregnancy multivitamin contains sufficient amounts of iron to lethally affect a young child. Most commonly sold pregnancy multivitamins and mineral supplements contain about 60 mg of elemental iron compound and are provided in 100 tablet bottles. This represents a total potential dose of 6000 mg or 6 grams. It is known that mild to moderate iron toxicity for toddlers starts as low as 20 to 60 mg/kg of body weight. 200-250 mg/kg of body weight is life threatening to lethal while at total ingestion of 6000 mg for a toddler will be lethal.

Surprisingly, little has been done in the prior art to address such terrible and preventable occurrence. So far, the prior art has provided micronutrient supplements, such as pregnancy multivitamins and mineral supplements, in bottles having childproof caps. However, if the mother does not screw a childproof cap tightly enough to engage the safety mechanism, a childproof cap will no longer be childproof.

### OBJECTS OF THE INVENTION

An object of the present invention is therefore to provide a novel micronutrient supplement dispensing package which combines a plurality of childproof features.

### SUMMARY OF THE INVENTION

More specifically, in accordance with an aspect of the present invention, there is provided a micronutrient supplement package provided in the form of a plurality of solid oral dosage units individually contained in blister packs wherein a portion of the dosage forms are iron-containing and at least half of the dosage units are essentially iron-free and wherein in a preferred embodiment the total amount of elemental iron in the package as sold to purchasers is less than about 1300 mg and most preferably 1050 mg or less of iron.

Other objects, advantages and features of the present invention will become more apparent upon reading of the following non-restrictive description of preferred embodiments thereof, given by way of example only with reference to the accompanying drawings.

### BRIEF DESCRIPTION OF THE DRAWINGS

Having thus generally described the invention, reference will now be made to the accompanying drawings, showing by way of illustration a preferred embodiment thereof, and in which:

FIG. 1 shows a perspective view of an example of a micronutrient supplement package of the present invention and more specifically an individual blister pack of a week's worth of the supplement of the present invention having an array of a first type of dosage unit which are iron-containing, to be taken at a given time of day, and an array of a second type of dosage unit which are essentially iron-free, to be taken at another time of day.

### DESCRIPTION OF THE PREFERRED EMBODIMENT

In a most preferred embodiment, the invention discloses a micronutrient supplement in the form of two distinct dosage units to be taken at spaced time intervals. In other words, the dosage unit is provided as a twice-a-day formulation which a different dosage units taken at each time interval.

As a first childproofing feature, the dispensing package contains blister wrapped and two distinct types of dosage units and preferably present in equal numbers. Each type of dosage unit contain different constituents with one dosage unit containing iron and other ingredients while the other dosage unit being essentially iron-free. Thus, if a child should accidentally ingest dosage units, the child would have a 50% chance to avoid ingesting iron.

An added benefit of the two dosage units is that calcium and iron ingredients may be placed in distinct and different dosage units so as to avoid their known propensity to mutually interfere with each other's absorption by the body.

In a most preferred embodiment, the two types of dosage units would be taken at spaced time intervals, e.g. one in the morning and one in the evening. In this most preferred embodiment, the compositions of the dosage units would be as follows:

### EXAMPLE 1

The following is an example of a morning dosage unit core formulation:

The following is an example of a morning dosage unit core formulation:

TABLE 1
Core ingredients:

| | | Label | | |
|---|---|---|---|---|
| Item # | Ingredient | Claim | | mg/Tab. |
| 1. | Beta-carotene | 2700 | IU | |
| 2. | Vitamin E | 30 | IU | |
| 3. | Vitamin C | 120 | mg | |
| 4. | Vitamin B₁ | 3 | mg | |
| 5. | Vitamin B₂ | 3.4 | mg | |
| 6. | Vitamin B₃ | 20 | mg | |
| 7. | Vitamin B₆ | 10 | mg | |
| 8. | Pantothenic Acid | 5 | mg | |
| 9. | Magnesium | 50 | mg | |
| 10. | Iodine | 0.15 | mg | |
| 11. | Iron | 35 | mg | |
| 12. | Copper | 2 | mg | |
| 13. | Zinc | 15 | mg | |
| 14. | Cross carmellose | | | 35 |
| | Sodium | | | |
| 15. | Sodium Lauryl | | | 3.5 |
| | Sulphate | | | |
| 16. | Microcrystalline | | | 180 |
| | Cellulose PH102 | | | |
| 17. | Starch 1500 | | | 55 |
| 18. | Magnesium | | | 3.5 |
| | Stearate | | | |

The following is an example of an evening dosage unit core formulation:

TABLE 2
Core ingredients:

| Item # | Ingredient | Label Claim | | Mg/Tab. |
|---|---|---|---|---|
| 1. | Vitamin D₃ | 250 | IU | |
| 2. | Calcium | 300 | mg | |
| 3. | Vitamin B₁₂ | 12 | mcg | |
| 4. | Folic Acid | 1.1 | mg | |
| 5. | Cross carmellose | | | 30 |
| | Sodium | | | |
| 6. | Sodium Lauryl | | | 3 |
| | Sulfate | | | |
| 7. | Magnesium | | | 3 |
| | Stearate | | | |

Dispensing Kit

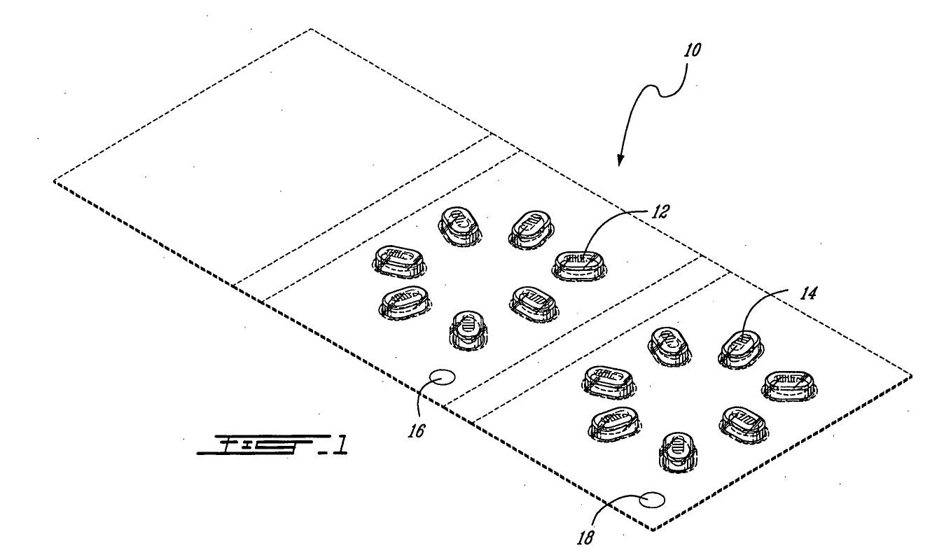

Referring now to FIG. 1, the preferred form of the present invention would be a dispensing kit containing two distinct dosage units grouped by type. Blister packs [10] of a week's worth of the supplement of the present invention having an array of blisters [12] of a first type of dosage unit to be taken at a given time of day and an array of blisters [14] of a second type of dosage unit to be taken at another time of day. Conveniently, 5 blister packs can be grouped in a box (not shown) for sale as monthly dosage packs. Advantageously, the package of dosage units will contain a 30 day supply, as four 7-day blister packs and one 2-day blister pack.

Still referring to FIG. 1, the blister pack includes graphical means [16] and [18] permitting a pregnant woman to differentiate between the morning and evening dosage types. These means may be, for example, a color code or diagrams surrounding a particular array of dosage units of the same type be it morning or evening.

An important benefit of the individual blisters [12] and [14] for each dosage unit is that a child who accidentally obtains access to blister packs will have to open each blister to get to a dosage unit. This is in contrast to prior devices where once access to a container of dosage units was obtained by a child such as by defeating the safety features of a childproof cap, the entire contents of dosage units became immediately available. Thus, this is the second childproofing feature.

Advantageously, each blisters [12] and [14] will be of the type having a clear plastic bubble sealed with aluminum foil. Still advantageously, the foil will be of a gauge which is difficult to pry open by a small child. This provides yet another line of defense in childproofing in the package of the present invention.

As indicated above, another line of childproofing feature in the package of the present invention is the fact that half of the dosage units are essentially iron-free. Thus, even if a child opens a blister, even chances will be that the dosage unit will be iron-free.

Yet another line of childproofing feature in the package of the present invention is the fact that the entire package contents of the present invention total less than about 1300 mg of elemental iron and most preferably 1050 mg or less (35 mg per dosage unit times 30 days), which is clearly a sub-lethal dose even for a toddler of, for example, 10 kg. The dose in such case would be 105 mg/kg of body weight. Thus, advantageously the dispensing package contains, in total, less than about 1050 to 1300 mg of elemental iron.

In contrast, a leading prenatal commercial preparation of multivitamins and mineral supplement currently sold in Canada is bottles of 100 tablets each containing 60 mg of elemental iron or 6000 mg of elemental iron. If the contents of the bottle were ingested, a 10 kg toddler would receive a lethal dose of 600 mg/kg of elemental iron.

Although the present invention has been described hereinabove by way of preferred embodiments thereof, it can be modified, without departing from the spirit and nature of the subject invention as defined in the appended claims.

1. A micronutrient supplement package comprising at least one solid oral unit dosage form comprising iron and at least one solid oral unit dosage form that is essentially iron free, wherein the unit dosage forms are individually comprised in blister packs, and wherein at least half of the unit dosage forms in the package are the essentially iron-free unit dosage form and wherein the total amount of elemental iron in the package is less than about 1300 mg of iron.

2. The micronutrient supplement package of claim 1 wherein the total amount of elemental iron is 1050 mg or less.

3. The micronutrient supplement package of claim 1 wherein the package comprises about a month's supply of the solid oral unit dosage forms and wherein the amount of elemental iron in each iron comprising dosage form is about 35 mg.

4. The micronutrient supplement package of claim 3 wherein the solid oral unit dosage forms are formulated as a twice-a-day dosage regimen with one dosage form comprising iron and the other dosage form being essentially iron-free.

5. A micronutrient supplement dispensing kit comprising at least one first type of solid oral unit dosage form comprising iron and at least one second type of solid oral unit dosage form that is essentially iron free, wherein said kit further comprises an array of foil-sealed blister cavities, wherein each blister cavity comprises the iron comprising unit dosage form or the essentially iron free unit dosage form, and wherein the number of the of essentially iron free unit dosage forms in the array is at least 50% of the total number of unit dosage forms in the array, said kit comprising a total amount of elemental iron less than about 1300 mg.

6. The kit of claim 5 wherein the total amount of elemental iron is 1050 mg or less.

7. The kit of claim 5 wherein the first type of unit dosage form is color-coded and the second type of unit dosage form is color-coded.

8. The kit of claim 5 wherein said kit comprises a monthly supply of unit dosage forms.

9. The kit of claim 8 wherein said kit comprises four separate 7-day blister arrays of unit dosage forms and one 2-day blister array of unit dosage forms and wherein said kit comprises dosage regimen instructions.

10. A prenatal or postpartum micronutrient supplement package comprising at least one solid oral unit dosage form comprising iron and at least one solid oral unit dosage form that is essentially iron free, wherein the unit dosage forms are individually comprised in blister packs, and wherein at least half of the unit dosage forms in the package are the essentially iron-free unit dosage form and wherein the total amount of elemental iron in the package is less than about 1300 mg of iron.

11. The prenatal or postpartum micronutrient supplement package of claim 10 wherein the total amount of elemental iron is 1050 mg or less.

12. The prenatal or postpartum micronutrient supplement package of claim 10 wherein the package comprises about a month's supply of unit dosage forms and wherein the amount of elemental iron in each iron comprising unit dosage form is about 35 mg.

13. The prenatal or postpartum micronutrient supplement package of claim 12, wherein the solid oral unit dosage forms are formulated as a twice-a-day dosage regimen with one dosage form comprising iron and the other dosage form being essentially iron-free.

14-18. (canceled)

19. The kit of claim 5, wherein the kit is a prenatal or postpartum micronutrient supplement dispensing kit, and wherein the kit comprises dosage regimen instructions for a prenatal or postpartum female.

20. The kit of claim 19, wherein the instructions comprise information on the recommended time of day for taking a unit dosage form of a first type and a unit dosage form of a second type.

21. The kit of claim 19 wherein the total amount of elemental iron is 1050 mg or less.

22. The kit of claim 19 wherein the first type of unit dosage form is color-coded and the second type of unit dosage form is color-coded.

23. The kit of claim 19 wherein said kit comprises a monthly supply of unit dosage forms.

24. The kit of claim 19 wherein said kit comprises four separate 7-day blister arrays of unit dosage forms and one 2-day blister array of unit dosage forms.
